(19) United States
(12) Patent Application Publication
(10) Pub. No.: US 2010/0297010 A1
(43) Pub. Date: Nov. 25, 2010

(54) TUMOR SUPPRESSOR GENE SCREENING USING RNA INTERFERENCE LIBRARIES AND METHOD OF TREATMENT

(76) Inventors: Anka Bric, Huntington Station, NY (US); Lars Zender, Hannover (DE); Cornelius Miething, Centerport, NY (US); Uli Bialucha, Huntington Station, NY (US); Scott W. Lowe, Cold Spring Harbor, NY (US)

Correspondence Address: WilmerHale/Cold Spring Harbor Laboratory, 399 Park Avenue, New York, NY, 10022, US

(21) Appl. No.: 12/600,427

(22) PCT Filed: May 16, 2008
(86) PCT No.: PCT/US08/06293

§ 371 Date: Jul. 14, 2010

Related U.S. Application Data
(60) Provisional application No. 60/930,532, filed on May 16, 2007.

(30) Foreign Application Priority Data
Feb. 8, 2008 (US) 61/065139

(51) Int. Cl. A61K 49/00; C12Q 1/68; A61K 31/7088; A61K 31/711; A61K 38/00; G01N 33/574; A61P 35/00

(52) U.S. Cl. 424/9.1; 435/6; 514/44 R; 514/44 A; 514/19.3; 435/7.23

(57) ABSTRACT

The present invention is directed to methods of identifying tumor suppressor genes in vivo, tumor suppressors thus found, methods of treatment taking advantage of the identified tumor suppressors, methods of and kits for diagnosis of cancer using the identified tumor suppressor, and pharmaceutical composition comprising an identified tumor suppressor or modulators thereof.

### RELATED APPLICATIONS

This application claims the benefit of U.S. Provisional Application 60/930,532, filed May 16, 2007, and U.S. Provisional Application 61/065,139, filed Feb. 8, 2008, the disclosure in which are herein incorporated by reference in its entirety.

### FIELD OF INVENTION

This invention relates to the use of RNA interference (RNAi) technology in vivo to efficiently identify genes that encode tumor suppressors by knocking out candidate genes using RNAi and observing whether tumors would develop.

### BACKGROUND

Cancer is the second leading cause of death in industrialized countries. It is well known that cancer arises from a combination of mutations in certain oncogenes and tumor suppressor genes. For example, Myc (cMyc) is a well-known proto-oncogene that affects or regulates apoptosis, cell differentiation, and stem cell self-renewal. Deregulation or overexpression of Myc is implicated in a wide range of human cancers and is often associated with aggressive, poorly differentiated tumors (Mo et al., 2006, Cell Cycle 5: 2191-2194). Conversely, p53, encoded by the Trp53 tumor suppressor gene, is a transcription factor that regulates the cell cycle by restricting cell proliferation in response to DNA damage or the deregulation of mitotic oncogenes. It is well known that mutations in or deletion of the Trp53 gene can result in tumorigenesis (Beraza, et al., 2007, Hepatology 45: 1578-1579; Schmitt et al., 1999, Genes Dev. 13: 2670-2677). These are but two examples of genetic causation of unregulated cell growth. Cancer may arise due to deregulation of many of the multiple points of cell cycle and cell differentiation system. Treatment and redifferentiation or destruction of cancerous tissues may be achieved more efficiently if the precise point of aberration is known for each instance of cancerous growth. However, despite the recent advances in elucidating the mechanism of tumorigenesis and development of treatment methods based on such understanding, the need for identifying genes involved in tumorigenesis remains urgent.

Investigation of the role of oncogenes or tumor suppressor genes in tumorigenesis can be facilitated by specifically silencing, or preventing from exerting its presence, the particular gene of interest. One such silencing means is through “RNA interference” or “RNAi.” RNAi stems from a phenomenon observed in plants and worms whereby double-stranded RNA (dsRNA) blocks gene expression in a specific and post-transcriptional manner. The dsRNA is cleaved by an RNAse III enzyme “DICER” into a 21-23 nucleotide small interfering RNA (siRNA). These siRNAs are incorporated into a RNA-induced silencing complex (RISC) that identifies and silences RNA complimentary to the siRNA. Without being bound by theory, RNAi appears to involve silencing of cytoplasmic mRNA by triggering an endonuclease cleavage, promoting translation repression, or possibly accelerating mRNA decapping (Valencia-Sanchez et al., 2006, Genes & Development 20: 515-524). Biochemical mechanisms of RNAi are currently an active area of research.

Three mechanisms of utilizing RNAi in mammalian cells have been described. The first is cytoplasmic delivery of siRNA molecules, which are either chemically synthesized or generated by DICER-digestion of dsRNA. These siRNAs are introduced into cells using standard transfection methods. The siRNAs enter the RISC complex to silence target mRNA expression.

The second mechanism is nuclear delivery, via viral vectors, of gene expression cassettes expressing a short hairpin RNA (shRNA). The shRNA is modeled on micro interfering RNA (miRNA), an endogenous trigger of the RNAi pathway (Lu et al., 2005, Advances in Genetics 54: 117-142, Fewell et al., 2006, Drug Discovery Today 11: 975-982). The endogenous RNAi pathway is comprised of three RNA intermediates: a long, largely single-stranded primary miRNA transcript (pri-miRNA), a precursor miRNA transcript having a stem-and-loop structure and derived from the pri-mRNA (pre-miRNA), and a mature miRNA. The miRNA gene is transcribed by an RNA polymerase II promoter into the pri-mRNA transcript, which is then cleaved to form the pre-miRNA transcript (Fewell et al., supra). The pre-miRNA is transported to the cytoplasm and is cleaved by DICER to form mature miRNA. miRNA then interacts with the RISC in the same manner as siRNA. shRNAs, which mimic pre-miRNA, are transcribed by RNA Polymerase II or III as single-stranded molecules that form stem-loop structures. Once produced, they exit the nucleus, are cleaved by DICER, and enter the RISC complex as siRNAs.

The third mechanism is identical to the second mechanism, except that the shRNA is modeled on pri-miRNA, rather than pre-miRNA transcripts (Fewell et al., supra). An example is the miR-30 miRNA construct (shRNAmir). The use of this transcript produces a more “physiological” shRNA that reduces toxic effects. The shRNAmir is first cleaved to produce shRNA, and then cleaved again by DICER to produce siRNA. The siRNA is then incorporated into the RISC for target mRNA degradation.

RNAi has been used to successfully identify and suppress target genes associated with tumorigenesis. For example, expression of microRNA-based shRNA specific to Trp53 produces “potent, stable, and regulatable gene knock-down in cultured cells . . . even when present at a single copy in the genome” (Dickins et al., 2005, Nature Genetics 37: 1289-1295). The tumors induced by the p53 knockdown regress upon re-expression of Trp53. Id. The suppression of the Trp53 gene expression by shRNA is also possible in stem cells and reconstituted organs derived from those cells (Hemann et al., 2003, Nature Genetics 33: 396-400). Moreover, the extent of inhibition of p53 function by the shRNA correlates with the type and severity of subsequent lymphomagenesis. Id.

However, there are conflicting views on which method of introducing and using RNAi mechanism is most effective. Some studies emphasize siRNA's several drawbacks, including transient effects, difficulty in delivery to nondividing primary cells, and concentration-dependent off-target effects. shRNAs expressed from viral vectors “are more versatile, allowing . . . stable integration, germline transmission, and the creation of in vivo animal models (Fewell et al., supra). shRNA is also more suitable for hard-to-transfect cells, due to its infection-based delivery, and has decreased concentration-dependent off-target effects. Id. In comparison with shRNA, shRNAmir is more efficiently processed into siRNA and produces a more consistent silencing of mRNA than shRNA. Id.

Despite the advantages of shRNA, other studies maintain that use of siRNA for RNAi purposes is emerging more rapidly than the use of shRNA (Lu et al., supra), partly because of the “increased effort required to construct shRNA expression systems before selection of active sequences and verification of biological activity are obtained.” Id. It is often time consuming and expensive to both construct shRNA expression cassettes and incorporate them into viral delivery systems. Id. On the contrary, use of synthetic oligonucleotides allows for rapid screening and studying of siRNA sequences and matching genes. Id. Moreover, recent studies investigating in vivo applications of RNAi focus on non-viral delivery of siRNA constructs as opposed to viral delivery of shRNA constructs, as viral vectors often raise concerns about safety and immunogenicity (Lu et al., supra; Vohies et al., 2007, Expert Rev. Anticancer Ther. 7: 373-382). In sum, there is no established method of RNAi that consistently produces the most effective RNA silencing.

Studies also vary in their use of genome-wide collections of pooled shRNA vectors versus small sets of shRNA vectors that target a specific gene family. The use of large shRNA libraries may lead to difficulties in measuring the relative abundance of each individual shRNA vector in a complex population of cells infected with thousands of vectors. In addition, the smaller scaled experiments allow “screening for relatively labor-intensive phenotypes.” Id. Pooled screens also pose several technological hurdles, such as obtaining uniform pools of viruses, creating robust design algorithms that enable gene knockdown at a single-copy level, and preventing large numbers of false positives (Fewell et al., supra). On the other hand, the use of barcodes, or unique sequence of nucleotides incorporated into each shRNA vector, allows for more accurate quantification of specific shRNAs in pooled analyses (Bernards et al., 2006, Nature Methods 3: 701-706). Moreover, larger shRNA library screens can be used to select for long-term phenotypes while smaller shRNA screens are mainly limited to “short-term” readouts. Id. Given the various benefits and drawbacks of both large and small scale screens, there is no suggestion that use of one method or the other is the most effective strategy for successful RNAi.

Finally, although certain tumor suppressors such as p53 are well-studied, the importance of other individual tumor suppressors is still unknown. As such, the extent of overall tumor suppressor gene loss required for maintaining tumors is poorly understood. Moreover, although there is potential to utilize Myc overexpression to investigate novel tumor suppressor genes, few scientists have so far been able to do so.

Established approaches for the investigation of novel tumor suppressor genes using RNAi are thus unavailable. As such, the invention described herein will further elucidate the mechanism of tumorigenesis and promote the development of treatment methods based on such understanding.

### SUMMARY OF THE INVENTION

The importance of individual tumor suppressors can be determined by silencing them in conjunction with a stimulus, such as oncogene expression or DNA damage. For example, it is well known that knockdown of p53 or ARF abrogates apoptosis, which can result in tumorigenesis. Knockdown of a tumor suppressor in cooperation with Myc overexpression in the mouse hematopoietic system will produce lymphomas, enabling the identification of a novel tumor suppressor gene by the appearance of a tumor and isolation and sequencing of the knocked-down gene from the tumor.

An aspect of the instant invention is a method of identifying a novel tumor suppressor gene by transfecting murine hematopoietic stem cells with a pool of shRNAs of candidate tumor suppressor genes, reconstituting the cells into mice, and identifying the shRNA from any tumor that develops. The shRNA is identified by isolating the genomic DNA from the tumor, amplifying the transfected shRNA by PCR, and sequencing the amplified DNA.

Another aspect of the invention is a method of identifying a therapeutic agent effective for treatment of cancer having no or diminished expression of certain tumor suppressor gene. Candidate agents are tested by contacting or introducing into the tumor arising from the shRNA targeting the tumor suppressor and determining whether the agents induce reduction of the tumor growth rate or regression of the tumor.

Another aspect of the invention is a method of treating cancer comprising the steps of determining the status in cancerous tissue of one or more of the tumor suppressor genes described herein or identified by the screening method described herein, and if any of the tumor suppressors is less abundant in cancerous tissue in comparison to the normal tissue, increasing the activity of said tumor suppressor(s).

In one embodiment, the less abundant tumor suppressor is increased by introducing the tumor suppressor into the cancerous tissue. In a particular embodiment, the tumor suppressor protein or a physiologically active fragment, analog, or mutant thereof is administered. In another particular embodiment, the tumor suppressor gene or a fragment or mutant thereof that encodes a physiologically active polypeptide is introduced into the cancer tissue and expressed. In yet another embodiment, known upstream factors of an identified tumor suppressor are modulated to increase the tumor suppressor expression. In another embodiment, known immediate downstream factors of an identified tumor suppressor are increased to augment the less abundant tumor suppressor.

Another aspect of the invention is a method of treating cancer comprising the steps of determining in cancerous tissue the activities of one or more tumor suppressor genes described herein or identified by the screening method described herein, the activities of which gene or genes are increased or decreased in comparison to the normal tissue, and administering a therapeutic agent that is known to be effective in treating such cancers that are associated with the increased or decreased activities of such gene or genes. Alternatively, an aspect of the invention is a method of treating cancer comprising the steps of determining in cancerous tissue the activities of one or more tumor suppressor genes described herein or identified by the screening method described herein, the activities of which gene or genes are decreased in comparison to the normal tissue, and administering a therapeutic agent that is known not to antagonize the gene or genes identified herein.

Yet another aspect of the invention is a pharmaceutical composition comprising a therapeutic agent for the treatment of cancer, which composition has specific utility to treat such cancer that has certain status regarding one or more tumor suppressors identified using the method described herein.

One embodiment of the invention is a pharmaceutical composition for the treatment of cancer in which the activity of said tumor suppressor is less than in normal tissue, comprising a tumor suppressor protein or a physiologically active fragment, analog, or mutant thereof. Another particular embodiment is a pharmaceutical composition for the treatment of cancer in which the activity of a tumor suppressor is less than in normal tissue, comprising a tumor suppressor gene or a fragment or mutant thereof that encodes a physiologically active polypeptide, to be introduced into the cancer tissue and expressed. In yet another embodiment, a pharmaceutical composition comprises one or more therapeutic agents that modulate known upstream factors of an identified tumor suppressor to increase the tumor suppressor expression.

Another aspect of the invention is a method of diagnosing a cancer in a subject. In one embodiment, the method comprises determining the biological activity of one or more tumor suppressor selected from those shown in Table I and comparing said activity to that in normal cells, wherein the subject is diagnosed with cancer if the activity of any one of tumor suppressor is substantially decreased or is not detectable. In another embodiment, the method comprises determining the expression of one or more tumor suppressor gene selected from genes shown in Table I and comparing said expression to that in normal cells, wherein the subject is diagnosed with cancer if said expression is substantially decreased or no expression is detected.

### BRIEF DESCRIPTION OF THE DRAWINGS

FIG. 1 shows the schematic of a tumor suppressor network.

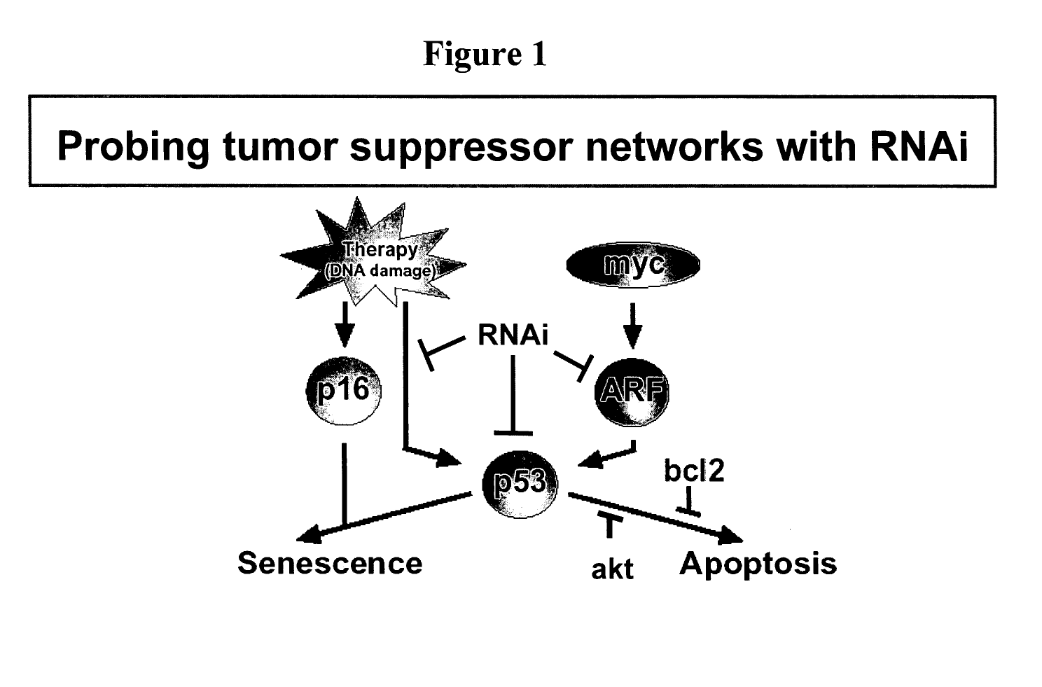

FIG. 2 is a schematic of shRNA library designs showing stem-loop configuration of shRNA.

FIG. 3 is a schematic of experimental procedure for identifying a tumor suppressor gene.

FIG. 4 is a schematic of an exemplary transfection vector.

FIG. 5 shows the survival rate of cells with knockdown of a tumor suppressor, Bim, with RNAi coupled with Myc over-expression.

FIG. 6 shows the survival curve when shRNA for p53 is introduced at dilutions down to 1/100.

FIG. 7 shows the fluorescence measurement from GFP, a marker for shRNA transfection, before and after reconstitution. The fluorescence of transfected HSCs before injection, and spleen cells and tumor cells after injection, are shown.

FIG. 8 is immunofluorescence of cells transfected with four different constructs of shRNA: The negative control consisted of shRNA to hCycD1 (sh hCycD1) a gene not present in the mouse genome. The), positive control consisted of shRNA to p53 (sh p53). The remaining two constructs consisted of a pool of several shRNAs (pool A16EH), and a yet-to-be identified gene (sh gene1), respectively.

FIG. 9 shows appearance of green tumors, i.e. tumors showing transfection with shRNA, in the various pools of shRNA tested.

FIG. 10 shows the schematic for validation procedure.

FIG. 11 shows an exemplary result of the in vitro validation of two tumor suppressor candidates (sh gene 1 and gene 2), a positive control (sh p53) and a negative control (sh control) at day 0 and day 4. The candidates scored just as well or better than the control, sh p53.

FIG. 12 shows the survival curves using the mouse lymphoma model of shRNA knockdowns of 5 probable tumor suppressor genes (Mek1; Angiopoietin 2 (Ang2); Rad17; Sfrp1; Numb).

### DETAILED DESCRIPTION OF THE INVENTION

The terms below, as used herein, have the following meaning.

An “analog” of a tumor suppressor is a molecule, which may be a peptide but can also be a structurally similar peptidomimetic, that has substantially similar physiological activities to the tumor suppressor. An analog can be a fragment of a full-length tumor suppressor, a mutant having one or more deletion, insertion, or substitution of amino acid residues within the polypeptide sequence, or a molecule composed partially or wholly of unnatural amino acids. An analog may also be a modified polypeptide having post translational modification, in vivo or in vitro.

As used herein, “antibody” means an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. Antibodies may also be modified by coupling them to other biologically or chemically functional moieties such as cross-linking agents or peptides.

“RNA interference,” or “RNAi” refers to a sequence-specific post-transcriptional gene silencing mechanism triggered by dsRNA, during which process the target RNA is degraded. RNA degradation occurs in a sequence-specific manner rather than by a sequence-independent dsRNA response, e.g., a PKR response.

“RNAi-expressing construct” or “RNAi construct” is a generic term which includes small interfering RNAs (siRNAs), shRNAs and shRNAmirs (see below), and other RNA species, and which can be cleaved in vivo to form siRNAs. “RNAi constructs” also include nucleic acid preparation designed to achieve an RNA interference effect, such as expression vectors capable of giving rise to transcripts which form dsRNAs or hairpin RNAs in cells, and/or transcripts which can produce siRNAs in vivo. Exemplary methods of making and delivering either long or short RNAi constructs can be found, for example, in WO01/68836 and WO01/75164.

A “short hairpin RNA (shRNA)” refers to a segment of RNA that is complementary to a portion of a target gene (e.g., complementary to one or more transcripts of a target gene), and has a stem-loop (hairpin) structure that can be used to silence gene expression. shRNA includes shRNAmir, which is miR-30 miRNA

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and the term is used consistently with its known meaning in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

The term “small molecule” refers to a compound having a molecular weight less than about 2500 amu, preferably less than about 2000 amu, even more preferably less than about 1500 amu, still more preferably less than about 1000 amu, or most preferably less than about 750 amu.

A “subject” or “patient” to be treated by the subject method can mean either a human or non-human animal.

As used herein, “treating” means either slowing, stopping or reversing the progression of the disorder. In a preferred embodiment, “treating” means reversing the progression to the point of eliminating the disorder or at least the symptoms of the disorder.

The term “vector” refers to a nucleic acid molecule capable of transporting another nucleic acid to which it has been linked. Nucleic acid vectors include, but are not limited to, plasmids, phagemids, viruses, other vehicles derived from viral or bacterial sources. These vectors are manipulated by the insertion or incorporation of both nucleic acid sequences expressing the precursor shRNA and free nucleic acid fragments which can be attached to these nucleic acid sequences. One type of nucleic acid vector is a plasmid, which refers to a circular double stranded DNA loop into which additional DNA segments may be ligated. A preferred type of vector for use in this application is a viral vector, wherein additional DNA segments may be ligated into a viral genome that is usually modified to delete one or more viral genes. Certain vectors are capable of autonomous replication in the host cell into which they are introduced (e.g., vectors having an origin of replication which functions in the host cell). Other vectors can be stably integrated into the genome of a host cell upon introduction into the host cell, and are thereby replicated along with the host genome.

The term “vehicle” is used to any molecule or structure capable of transporting nucleic acids, polypeptides, small molecules, and other physiologically relevant compositions into a location in a subject in vivo or into a cell in such a way that the transported composition carry out biologically relevant activity after having reached such location. A vehicle may be lipid, carbohydrate including polysaccharide, poly-amino acid, ionophore, cationic or anionic detergent, or any chemical composition of various sizes and, preferably with no or low toxicity to the subject or the cell.

“Transfection” or “infection” means introduction into a live cell, either in vitro or in vivo, certain nucleic acid construct, preferably into a desired cellular location of the cell, and is functional. Such presence of the introduced nucleic acid may be stable or transient. Successful transfection or infection will have an intended effect in the transfected cell, such as silencing or enhancing a gene target, or triggering target physiological event.

RNAi has been widely used to silence or inhibit the expression of a target gene. RNAi is a sequence-specific post-transcriptional gene silencing mechanism triggered by dsRNA. It causes degradation of mRNAs homologous in sequence to the dsRNA. The mediators of the degradation are 21-23-nucleotide siRNAs generated by cleavage of longer dsRNAs by DICER, a ribonuclease III-like protein. Molecules of siRNA typically have 2-3-nucleotide 3′ overhanging ends resembling the RNAse III processing products of long dsRNAs that normally initiate RNAi. When introduced into a cell, they assemble an endonuclease complex (RISC), which then guides target mRNA cleavage. As a consequence of degradation of the targeted mRNA, cells illustrating the specific phenotype associated with the suppression of the corresponding protein product are obtained. If the protein that is knocked down possesses an activity that attenuates cell growth, such knock down will result in unbridled growth of the cells.

The small size of siRNAs, compared with traditional antisense molecules, prevents activation of the dsRNA-inducible interferon system present in mammalian cells. This helps avoid the nonspecific phenotypes normally produced by dsRNA larger than 30 base pairs in somatic cells. See, e.g., Elbashir et al., 2002, Methods Enzymol. 26: 199-213; McManus and Sharp, 2002, Nature Reviews 3: 737-747; Hannon, 2002, Nature 418: 244-251; Brummelkamp et al., 2002, Science 296: 550-553; Tuschl, 2002, Nature Biotechnology 20: 446-448; U.S. Application US2002/0086356 A1; WO 99/32619; WO 01/36646; and WO 01/68836.

RNAi is also possible via gene expression cassettes expressing shRNA or shRNAmir. shRNA and shRNAmir are modeled on intermediate constructs of miRNA. Both are cleaved by DICER to form siRNAs and interact with the RISC complex in the same manner as siRNA.

shRNA-mediated knockdown of p53 (Hemann et al., 2003, Nature Genetics. 33: 396-400. Epub 2003 Feb. 3) or Bim (Dickins et al., 2005, Nature Genetics 37: 1289-95. Epub 2005 Oct. 2) has been shown to cause lymphomas. This mouse lymphoma model is useful to screen for potential tumor suppressors. By infecting the hematopoietic stem cells (HSCs) with pools of shRNAs rather than single constructs, the system can be used to screen for several novel tumor suppressor genes. The appearance of a tumor indicates that a tumor suppressor gene has been knocked down. From each pool, one or several genes are expected to be identified whose knockdown result in lymphoma. From the tumors that arise, genomic DNA is isolated, and the integrated hairpins are amplified using polymerase chain reaction, cloned back into a vector, and then identified by sequencing.

In a preferred embodiment, the shRNAs useful for this method are designed based on an endogenous miRNA and are driven by an RNA polymerase II promotor. Such shRNA can introduced into the HSCs using retroviral vectors for infection.

### Useful Forms of RNAi Reagents

Libraries. In one embodiment, the pools of shRNA useful to practice the method of the instant invention comprise a library that was named “the Cancer 1000,” which was constructed by Steve Elledge and Greg Hannon. The “Cancer 1000” shRNA library includes a mixture of well characterized oncogenes and tumor suppressor genes in addition to many poorly-characterized genes somehow related to cancer, across many ontological groups, as compiled by literature mining. In another embodiment, the pools of shRNA useful to practice the method of the instant invention come from a cDNA library that includes a mixture of oncogenes. A similar library design rationale may be easily applied to construct RNAi libraries targeting genomes of other organisms, such as the human. Examples of known tumor suppressors are p53, BRCA1, BRCA2, APC, p16^{INK4a}, PTEN, NF1, NF2, and RB1. These known tumor suppressors are expected to be identified and can serve as positive controls. Negative controls can include shRNAs to genes not present in the organism's genome or empty vectors.

shRNA and miRNA. When a nucleic acid construct encoding a short hairpin RNA is introduced into a cell, the cell incurs partial or complete loss of expression of the target gene. In this way, a short hairpin RNA functions as a sequence-specific expression inhibitor or modulator in transfected cells. The use of short hairpin RNAs facilitates the down-regulation of the target gene and allows for analysis of hypomorphic alleles. Short hairpin RNAs useful in the invention can be produced using a wide variety of well known RNAi techniques. The invention may be practiced using short hairpin RNAs that are synthetically produced as well as microRNA (miRNA) molecules that are found in nature and can be remodeled to function as synthetic silencing short hairpin RNAs. DNA vectors that express perfect complementary short hairpin RNAs (shRNAs or shRNAmirs) are commonly used to generate functional siRNAs.

In preferred embodiments, the siRNA useful to practice the invention or a precursor molecule thereof, may be a shRNA or a shRNAmir, both modeled on miRNA intermediates. In description of the invention and examples below, where shRNA is recited, it is a preferred embodiment but not exclusive, and other forms of siRNA are contemplated. shRNA and shRNAmir are sequences of RNA that make tight hairpin turns (stem-loop structure) that can be used to silence gene expression. miRNAs are single-stranded RNA molecules of about 21-23 nucleotides and are part of an endogenous RNAi system. miRNAs are usually processed from two RNA intermediates: a primary miRNA (pri-miRNA) transcript and a precursor miRNA (pre-miRNA). The precursor transcripts are converted into short stem-loop structures, and then to functional miRNAs. Many miRNA intermediates can be used as models for shRNA or shRNAmir, including without limitation a miRNA comprising a backbone design of miR-15a, -16, -19b, -20, -23a, -27b, -29a, -30b, -30c, -104, -132s, -181, -191, -223. See US 2005-0075492 A1 (incorporated herein by reference).

MicroRNAs (miRNAs) are endogenously encoded RNAs that are about 22-nucleotide-long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). Importantly, miRNAs are expressed in a highly tissue-specific or developmentally regulated manner, and this regulation is likely key to their predicted roles in eukaryotic development and differentiation. Analysis of the endogenous role of miRNAs will be facilitated by techniques that allow the regulated over-expression or inappropriate expression of authentic miRNAs in vivo. Their ability to regulate the expression of siRNAs will greatly increase their utility both in cultured cells and in vivo. Thus, one can design and express artificial miRNAs based on the features of existing miRNA genes, such as the gene encoding the human miR-30 miRNA. These miR30-based shRNAs and shRNAmirs have complex folds, and, compared with simpler stem/loop style shRNAs, are more potent at inhibiting gene expression in transient assays. Moreover, they are associated with less toxic effects in cells.

miRNAs are first transcribed as part of a long, largely single-stranded primary transcript (pri-miRNA) Lee et al., 2002, EMBO J. 21: 4663-4670). This pri-miRNA transcript is generally, and possibly invariably, synthesized by RNA polymerase II and therefore is normally polyadenylated and may be spliced. It contains an ˜80-nt hairpin structure that encodes the mature ˜22-nt miRNA as part of one arm of the stem. In animal cells, this primary transcript is cleaved by a nuclear RNaseIII-type enzyme called Drosha (Lee et al., 2003, Nature 425: 415-419) to liberate a hairpin miRNA precursor, or pre-miRNA, of ˜65 nt. This pre-miRNA is then exported to the cytoplasm by exportin-5 and the GTP-bound form of the Ran cofactor (Yi et al., 2003, Genes & Development 17: 3011-3016). Once in the cytoplasm, the pre-miRNA is further processed by Dicer, another RNaseIII enzyme, to produce a duplex of ˜22 by that is structurally identical to an siRNA duplex (Hutvagner et al., 2001, Science 293: 834-838). The binding of protein components of the RNA-induced silencing complex (RISC), or RISC cofactors, to the duplex results in incorporation of the mature, single-stranded miRNA into a RISC or RISC-like protein complex, while the other strand of the duplex is degraded (Bartel, 2004, Cell 116: 281-297).

The miR-30 architecture can be used to express miRNAs or siRNAs from RNA polymerase II promoter-based expression plasmids. See also Zeng et al., 2005, Methods Enzymol. 392: 371-380 (incorporated herein by reference).

In some instances the precursor miRNA molecule may include more than one stem-loop structure. The multiple stem-loop structures may be linked to one another through a linker, such as, for example, a nucleic acid linker, a miRNA flanking sequence, other molecule, or some combination thereof.

In certain embodiments, useful interfering RNAs can be designed with a number of software programs, e.g., the OligoEngine siRNA design tool available at www.oligoengine.com. The siRNAs of this invention may be about, e.g., 19-29 base pairs in length for the double-stranded portion. In some embodiments, the siRNAs are shRNAs having a stem of about 19-29 base pairs and a nucleotide loop of about 4-34 bases. Preferred siRNAs are highly specific for a region of the target gene and may comprise a 19-29 base pair fragment of the mRNA of a target gene, with at least one, but preferably two or three, base pair mismatch with a nontarget gene-related sequence. In some embodiments, the preferred siRNAs do not bind to RNAs having more than three base pair mismatches with the target region.

In certain embodiments, artificial miRNA constructs based on miR-30 (microRNA 30) may be used to express precursor miRNA/shRNA. For example, Silva et al., 2005, Nature Genetics 37: 1281-88, have described extensive libraries of pri-miR-30-based retroviral expression vectors that can be used to down-regulate almost all known human (at least 28,000) and mouse (at least 25,000) genes (see RNAi Codex, a single database that curates publicly available RNAi resources, and provides the most complete access to this growing resource, allowing investigators to see not only released clones but also those that are soon to be released, available at http://codex.cshl.edu). Although such libraries are driven by RNA polymerase III promoters, they can be easily converted to the subject RNA polymerase II-driven promoters (see the Methods section in Dickins et al., 2005, Nature Genetics 37: 1289-95; also see page 1284 in Silva et al., 2005 supra).

In certain embodiments, the subject precursor miRNA cassette may be inserted within a gene encoded by the subject vector. For example, the subject precursor miRNA coding sequence may be inserted within an intron, the 5′- or 3′-UTR of a reporter gene, etc.

Other methods of RNAi may also be used in the practice of this invention. See, e.g., Scherer and Rossi, 2003, Nature Biotechnology 21: 1457-65 for a review on sequence-specific mRNA knockdown using antisense oligonucleotides, ribozymes, DNAzymes. See also, International Patent Application PCT/US2003/030901 (Publication No. WO 2004-029219 A2), filed Sep. 29, 2003 and entitled “Cell-based RNA Interference and Related Methods and Compositions.” See also Fewell et al., supra, for a description of inducible shRNA, in which the vector does not express the shRNA unless a specific reagent is added. Several studies investigating the function of essential genes using RNAi rely on inducible shRNA. For example, shRNAmir constructs can be created based on a tetracycline-responsive promotor system, such that shRNA expression is regulated by changing doxycycline levels.

Vector. In an embodiment of the present invention, a pool of shRNAs is introduced into murine HSCs from Eμ-myc mice, using a vector known in the art. In certain embodiments, the vector is a viral vector. Exemplary viral vectors include adenoviral vectors, lentiviral vectors, or retroviral vectors. Many established viral vectors may be used to transfect foreign constructs into cells. The definition section below provides more details regarding the use of such vectors.

To facilitate the monitoring of the target gene knockdown, and the formation and progression of the cancer, cells harboring the RNAi-expressing construct may additionally comprise a marker construct, such as a fluorescent marker construct. The marker construct may express a marker, such as green fluorescent protein (GFP), enhanced green fluorescent protein (EGFP), Renilla Reniformis green fluorescent protein, GFPmut2, GFPuv4, yellow fluorescent protein (YFP), enhanced yellow fluorescent protein (EYFP), cyan fluorescent protein (CFP), enhanced cyan fluorescent protein (ECFP), blue fluorescent protein (BFP), enhanced blue fluorescent protein (EBFP), citrine and red fluorescent protein from discosoma (dsRED). Other suitable detectable markers include chloramphenicol acetyltransferase (CAT), luciferase lacZ (β-galactosidase), and alkaline phosphatase. The marker gene may be separately introduced into the cell harboring the shRNA construct (e.g., co-transfected, etc.). Alternatively, the marker gene may be linked to the shRNA construct, and the marker gene expression may be controlled by a separate translation unit under an IRES (internal ribosomal entry site). In a preferred embodiment, the marker is a green fluorescent protein (GFP).

To facilitate the quantification of specific shRNAs in a complex population of cells infected with an entire library of shRNAs, each shRNA construct may additionally comprise a barcode. A barcode is a unique nucleotide sequence (generally 19-mer), linked to each shRNA. The barcode can be used to monitor the abundance of each shRNA via micoarray hybridization (Fewell et al., supra). In a preferred embodiment, each shRNA construct also comprises a unique barcode. For more information on the use of barcodes in shRNA pooled analyses, see Bernards et al., 2006, Nature Methods 3: 701-706, and Chang et al., 2006, Nature Methods 3: 707-714.

### Outline of the Process.

Transfection. Animals useful for the practice of the present invention overexpress Myc. Such animals can be rodents, for example, mice. The myc gene can be under the control of a promoter/enhancer region specific to B cells, such that the myc gene is specifically expressed in B cells, for example Eμ-myc (Adams, J. M. et al., Nature 318:533-538, 1985). Eμ-myc/tumor suppressor gene mutation mice are mice having the genotype of the myc oncogene, under the control of the EA IgH enhancer, in combination with a tumor suppressor gene mutation whose presence results in an increase in the probability of the development of tumors in an animal or human (relative to the probability of tumor development in animals in which wild type alleles of the suppressor gene are present). The myc gene can be one as described in Harris, A. W., J. Exp. Med. 167:353-371 (1988) or the allelle described by Langdon, W. Y. et al., Cell 47:11-18 (1986), for example. The myc gene can also be a naturally-occurring gene, either cellular or viral, a natural variant or an artificially altered variant of myc. HSCs from such Eμ-myc mice are transfected with a pool of siRNAs, preferably shRNAs, targeting candidate tumor suppressor genes, and the transfected cells are reconstituted into mice. Mice receiving cells transfected with tumor suppressor knockdowns, in cooperation with overexpression of Myc, develop tumors, recognizable by the green fluorescence. From the tumors that arise, genomic DNA is isolated, and the integrated hairpins are amplified using polymerase chain reaction, cloned back into a vector, and then identified by sequencing. Methods for such isolation, amplification, cloning and sequencing is well known in the art.

Various methods well known to one skilled in the art may be used to determine the growth or viability of recipient cells expressing an RNAi-expressing construct in vitro. Such assays may be conducted using commercially available assay kits or methods well known to one or ordinary skill in the art. For example, cell viability can be determined by MTT assay or WST assay, a standard colorimetric assay for measuring cellular growth. The effect of the target gene knockdown can also be determined using cellular proliferation assays or cellular apoptosis/necrosis assays. In vitro cellular proliferation assays can be performed by determining the amount of cells in a culture over time. Cell numbers may be evaluated using standard techniques. Cellular apoptosis can be measured, for example, using a commercial apoptosis assay kit such as VYBRANT Apoptosis Assay Kit #3 (Molecular Probes). Cells can also be stained with P1 or DAP1 to detect apoptotic nuclei.

In certain embodiments, recipient cells expressing an RNAi construct (e.g., a shRNA) against a target gene are sorted based on a selectable marker whose expression substantially matches the expression of the RNAi molecule. In one exemplary embodiment, the selectable marker is fluorescence-based. In one exemplary embodiment, the selectable marker is GFP. In one embodiment, cells harboring the selectable marker are sorted using fluorescence-activated cell sorting (FACS). FACS is a powerful system which not only quantifies the fluorescent signal but also separates the cells that contain preselected characteristics (such as fluorescence intensity, size and viability) from a mixed population. Laser light is directed at individual cells as they flow through the FACS. A light scatter pattern is generated when the dense nuclear material of the cell interferes with the path of the laser beam.

Recipient cells expressing an RNAi construct (e.g., a shRNA) against a target gene may be subsequently transplanted into a recipient non-human animal. Alternatively, after shRNA infection, the cells may be injected subcutaneously into a recipient non-human animal. The size and growth of tumors in the recipient, the survival of tumor-free recipients, and overall survival of the recipient may then be observed to investigate the effect of target-gene-knockdown in vivo. The size and growth of tumors may be examined by any of many known methods in the art, such as histological methods, immunohistochemical methods, TUNEL-staining, etc. In certain embodiments, the non-human animal is a mouse. In certain embodiments, the recipient animal is an immuno-compromised animal, such as a nude mouse.

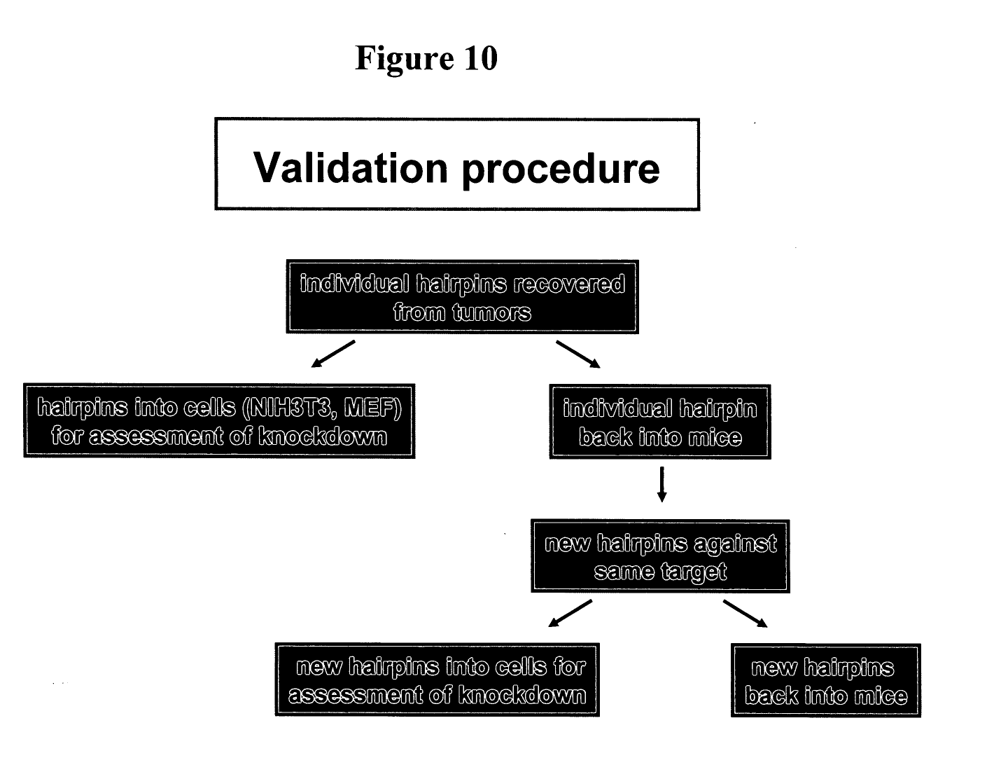

Validation. Identified siRNAs are validated by introduction into cells and assessment for knockdown, which is done by immunoblotting or QPCR. The general scheme of validation procedure is shown in FIG. 10. If positive, the individual hairpins are further evaluated for their activities in mice. To confirm the involvement of the target gene, new hairpins are created against the same gene and put back into mice to rule out off-target effects. These newly created hairpins are evaluated through knockdown as well.

Knockdown of single siRNA candidates, as analyzed by survival curves indicate that they result in tumorigenesis. The candidate tumor suppressors are further assessed by in vitro validation processes to ascertain the mechanism by which knockdown of these putative tumor suppressors is tumorigenic. Such processes will elucidate whether the tumorigenesis is due to apoptotic defects or proliferation advantage. For example, response to growth factor withdrawal, DNA damage response to cytotoxic drugs, or activity of downstream targets would be further examined. In addition, deletions or mutations in human tumors can be explored and compared, using, for example, the ROMA database and human tumor samples.

### Method of Screening

The identified shRNA targeting tumor suppressors are useful for screening therapeutic agents. One aspect of the invention is a method for testing a lymphoma arising from an Eμ-myc/shRNA tumor suppressor-transfected lymphoma for sensitivity to a treatment. Lymphoma cells are cultured in vitro, a treatment is administered to the cells (e.g., a drug is contacted with the cells), and the cells can be monitored for growth (e.g., by observing cell number, confluence in flasks, staining to distinguish viable from nonviable cells). A failure to increase in viable cell number, a slower rate of increase in cell number, or a decline in viable cell number, compared to cells which have been left untreated, or which have been mock-treated, is an indication of sensitivity to the treatment.

The treatment to be tested can be one or more substances, for example, a known anti-cancer agent, such as adriamycin, cylophosphamide, prednisone, vincristine or a radioactive source. The treatment can also be exposure to various kinds of energy or particles, such as gamma-irradiation, or can be a combination of approaches. In some cases, the treatment can also be administration of one or more substances or exposure to conditions, or a combination of both, wherein the effects of the treatment as anti-cancer therapy are unknown. Candidate agents may be further tested in lymphoma tumors in situ in a mouse. Animals can be tested essentially as described in U.S. Pat. No. 6,583,333. Briefly, Eμ-myc transgenic mice are treated with maximum tolerated dose of a candidate therapeutic agent (for example, 10 mg/kg body weight) by intraperitoneal injection. Treated mice were monitored for remission and for relapse by palpation and by blood smears to obtain white blood cell counts. Palpation is performed by gently feeling the mouse for bumps under the skin, which are enlarged lymph nodes. Blood smears are done by collecting blood from the tail artery, and examining a dried droplet of blood which has been smeared on a glass microscope slide to be one cell layer thick at the edge. The blood smear is stained after drying, using LEUKOSTAT™ stain (Fisher Diagnostics cat. #CS43A-C). The blood smear can be mounted with Permount™ histological mounting medium (Fisher Scientific). Slides are viewed under 40× or oil emersion. Blood from mice affected by lymphoma are always compared with blood from mice from a normal mouse drawn at the same time.

### Method of Diagnosis

Another aspect of the invention is a method of diagnosing a cancer in a subject. In one embodiment, the method comprises obtaining a tissue sample from the subject, determining the biological activity of one or more tumor suppressor selected from those shown in Table I in the tissue sample and comparing said activity to that in normal tissue, wherein the subject is diagnosed with cancer if the activity of any one of tumor suppressor is substantially decreased or is not detectable in the tissue sample. In another embodiment, the method comprises determining the expression of one or more tumor suppressor gene selected from genes shown in Table I in the tissue sample and comparing said expression to that in normal tissue, wherein the subject is diagnosed with cancer if said expression is substantially decreased or no expression is detected in the tissue sample. The biological sample of the present invention can be any sample suitable for the methods provided by the present invention. In one embodiment, the biological sample of the present invention is a tissue sample, e.g., a biopsy specimen such as samples from needle biopsy. In another embodiment, the biological sample of the present invention is a sample of bodily fluid, e.g., serum, plasma, urine, and ejaculate. Normal tissue used as negative control can be tissue from any individual not diagnosed with cancer and of the same species as the subject. Such subject does not show any symptoms or known biological marker for the cancer being tested for. Preferably, the quantitative measurement from the tissue sample is compared to the values obtained from more than one normal tissue.

The tumor suppressors described herein are detectable using monoclonal antibodies prepared using methods known in the art. The tumor suppressor genes described herein are detectable using various methods available in the art, including quantitative PCR.

Another aspect of the present invention is a kit useful for identifying cancerous transformation in a cell or tissue, e.g., using the decrease or lack of a tumor suppressor gene identified herein. In one embodiment, the present invention provides a kit, e.g., a compartmentalized carrier including a first container containing a pair of primers for amplification of a tumor suppressor, a second container containing a pair of primers for amplification of a region in a reference gene, and a third container containing a first and second oligonucleotide probe specific for the amplification of the biomarker and the region of the reference gene, respectively. A reference gene may be any gene that is consistently expressed in any tissue regardless of whether the tissue is cancerous.

### Method of Treatment

Another aspect of the invention is a method of treating cancer comprising the steps of determining the status in cancerous tissue of one or more of the tumor suppressor genes described herein or identified by the screening method described herein, and if any of the tumor suppressors is less abundant in cancerous tissue in comparison to the normal tissue, increasing the activity of said tumor suppressor(s).

In one embodiment, the less abundant tumor suppressor is increased by introducing the tumor suppressor into the cancerous tissue. In a particular embodiment, the tumor suppressor protein or a physiologically active fragment, analog, or mutant thereof is administered. The administration dosage is determined by titer so that the amount of tumor suppressor protein is about the same as that of normal tissue. In another particular embodiment, the tumor suppressor gene or a fragment or mutant thereof that encodes a physiologically active polypeptide is introduced into the cancer tissue by means of a vector and expressed. Examples of vectors useful for this method are based on adenovirus (Ad), adeno-associated virus (AAV), herpes simplex virus type 1-derived vectors (HSV-1), and retrovirus/lentivirus vectors. Adenovirus and lentivirus based gene therapy systems have already been used in human trials with success. Other types of vehicles useful for gene delivery are non-viral vehicle systems using cationic lipids, polymers, or both as carriers. An example is polyethylenimine (PEI) assisted delivery. For useful vectors and vehicles, see, for example, Vector Targeting for Therapeutic Gene Delivery, eds. Curiel and Douglas, Wiley-Liss, 2002. The suppressor genes may be expressed by operably linking the gene to an inducible promoter, for example radiation-sensitive promoters, including VEGF, Rec-A, and WAF-1 promoters. Alternatively, tetracycline inducible expression systems may be suitable in certain instances. In yet another embodiment, known upstream factors of an identified tumor suppressor is modulated to increase the tumor suppressor expression.

More specifically, an embodiment of the invention is a method for treating cancer comprising the steps of determining the status in cancerous tissue of one or more of the tumor suppressor genes described in Table I of Example 3, and if any of the tumor suppressors is less abundant in cancerous tissue in comparison to the normal tissue, increasing the activity of said tumor suppressor(s). In certain embodiments, said tumor suppressor gene for which the status is determined is selected from MEK1; Angiopoietin2 (Ang2); Rad17; Sfrp1; and Numb. A known upstream factor for MEK1, for example, is Raf kinase. Thus, one embodiment of the invention is modulating Raf kinase activity specifically to modulate MEK1 activity. The immediate downstream factor of MEK1 is Erk1 and Erk2. Thus, yet another example of the invention is increasing Erk1 and Erk2 activities to compensate for low MEK1 activity.

Another aspect of the invention is a method of treating cancer comprising the steps of determining in cancerous tissue the activities of one or more tumor suppressor genes described herein or identified by the screening method described herein, the activities of which gene or genes are decreased in comparison to the normal tissue, and administering a therapeutic agent that is known to be effective in treating such cancers that are associated with the decreased activities of such gene or genes. Alternatively, an aspect of the invention is a method of treating cancer comprising the steps of determining in cancerous tissue the activities of one or more tumor suppressor genes described herein or identified by the screening method described herein, the activities of which gene or genes are decreased in comparison to the normal tissue, and administering a therapeutic agent that is known not to antagonize the gene or genes identified herein.

More particularly, an embodiment of this aspect of the invention can be practiced using the tumor suppressor genes listed in the Table I of Example 3, or any other genes that are identified using the screening method described herein. More particularly, said tumor suppressor gene for which the status is determined is selected from MEK1; Angiopoietin2 (Ang2); Rad17; Sfrp1; and Numb.

### Pharmaceutical Composition

Yet another aspect of the invention is a pharmaceutical composition comprising a therapeutic agent for the treatment of cancer, which composition has specific utility to treat such cancer that has certain status regarding one or more tumor suppressors identified using the method described herein.

One embodiment of the invention is a pharmaceutical composition for the treatment of cancer in which the activity of said tumor suppressor is decreased compared in normal tissue, comprising said tumor suppressor protein or a physiologically active fragment, analog, or mutant thereof.

Another particular embodiment is a pharmaceutical composition for the treatment of cancer in which the activity of a tumor suppressor is decreased compared to in normal tissue, comprising a vector containing the tumor suppressor gene or a fragment or mutant thereof that encodes a physiologically active polypeptide, wherein such vector is introduced into the cancer tissue and the tumor suppressor or its fragment or mutant is expressed. In yet another embodiment, a pharmaceutical composition comprises one or more therapeutic agents that modulate known upstream factors of an identified tumor suppressor to increase the tumor suppressor expression. Another embodiment is a pharmaceutical composition comprising one or more therapeutic agents that modulate, or that are, known immediate downstream factors of an identified tumor suppressor to augment the decreased expression of the tumor suppressor.

More particularly, an embodiment of this aspect of the invention can be practiced using the tumor suppressor genes listed in the Table I of Example 3, or any other genes that are identified using the screening method described herein. More particularly, said tumor suppressor gene for which the status is determined is selected from MEK1; Angiopoietin2 (Ang2); Rad17; Sfrp1; and Numb.

### EXAMPLES

### Example 1

### Selecting an RNAi Library

To identify a gene whose inactivation in a cancer cell results in the cancer cell's resistance to an apoptotic-inducing cancer drug, it is important to choose a suitable RNAi library. A genome-wide screening library, with shRNA constructs representing each open reading frame, may be used. Alternatively, one may choose a single shRNA construct or a very small RNAi library of known biological function.

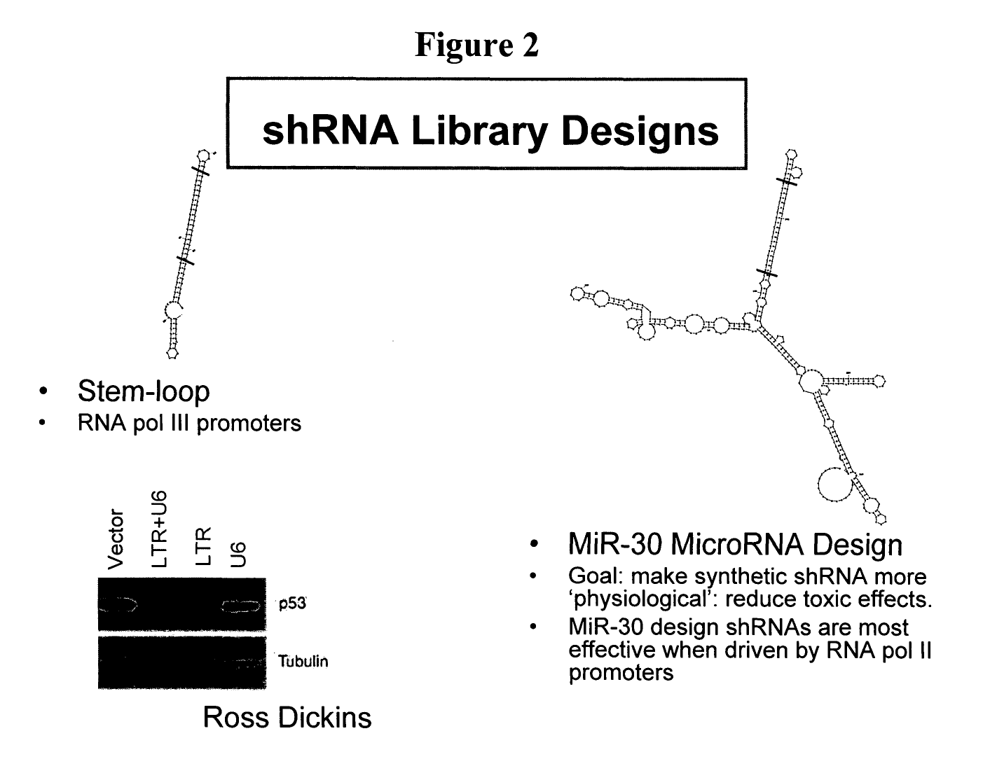

FIG. 2 is a schematic of shRNA library designs showing stem-loop configuration of shRNA. The shRNA design was based on an endogenous miRNA construct, miR-30, that is driven by a RNA polymerase II promotor. One screening was performed using the “Cancer 1000” shRNA subset containing about 2300 shRNAs targeting about 1000 mouse genes. The“Cancer 1000” shRNA library includes a mixture of well characterized oncogenes and tumor suppressor genes, in addition to many poorly-characterized genes, across many ontological groups, as compiled by literature mining (Harvard Institute of Proteomics). This library represented a balance between the relatively narrow biology of small, functional gene sets and a genome-wide screening. Another screening for oncogenes was performed using a cDNA library.

In this particular example, the RNAi libraries of choice were the Hannon-Elledge shRNA library (Silva et al., 2005, Nature Genetics 37: 1281-1288), cDNA library targeting oncogenes. In this particular example, the RNAi library of choice was the Hannon-Elledge shRNA library (Silva et al, 2005 supra), administered to lymphoma cells via retroviral infection. The stable integration and knockdown via retroviral constructs, even at single copy (Dickins et al., 2005, Nature Genetics 37: 1289-1295), allows for longer term experiments and easier shRNA construct recovery than transfection-based techniques.

In one example, 2352 shRNAs in total were prepared for testing. shRNAs were grouped into 49 pools, each of which contained 48 shRNAs. One pool was introduced into three mice. As a positive control, shRNA against p53 was used, and as negative controls, an empty vector and a shRNA against hCycD1, which has no target in the mouse genome, were used.

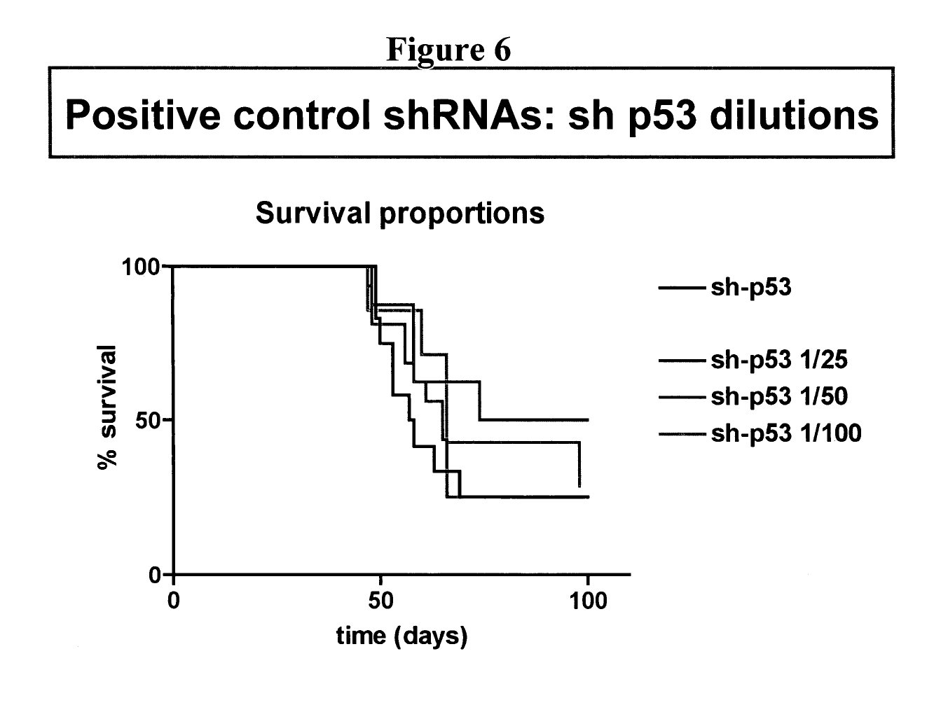

As preliminary experiments, various dilutions of sh p53 were tested to ascertain effective pool size. FIG. 6 shows the results of the dilution experiments. Dilution as low as 1/100 were effective for sh p53, exhibiting nearly as strong an effect as undiluted sh p53. Taking into consideration that p53 is a very powerful tumor suppressor and therefore no hit is likely to be as strong as p53, pools of about 50 shRNA were chosen as a suitable pool size and can expected to produce some genuine hits, at the same time resulting in a manageable number of pools.

In order to facilitate the monitoring of infection efficiency and tumor progression, green fluorescent protein (GFP) was used as marker for the shRNAs. FACS analysis for GFP showed the enrichment of certain shRNAs throughout the experiment.

### Example 2

### Vector Construction and Results of Reconstitution

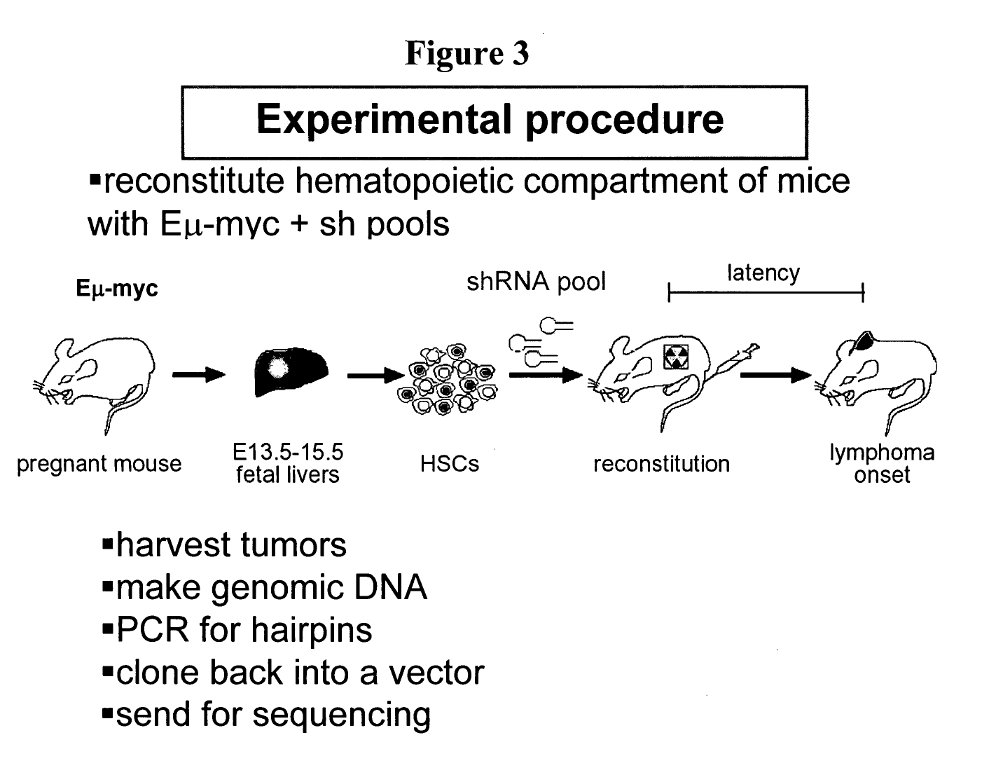

FIG. 3 is a schematic of experimental procedure for identifying a tumor suppressor gene. Briefly, Myc was over-expressed in the murine hematopoietic stem cells (HSCs). The murine HSCs were transfected with shRNA via vectors and then reconstituted into mice. Tumors that developed within sixteen weeks of reconstitution were examined. Tumors that developed six months after reconstitution were determined to be standard Eμ-myc lymphomas. The genomic DNA from the tumors that develop is isolated, and the shRNA expressed in the cell is amplified using PCR. The shRNA is then cloned back into a vector and identified by sequencing.

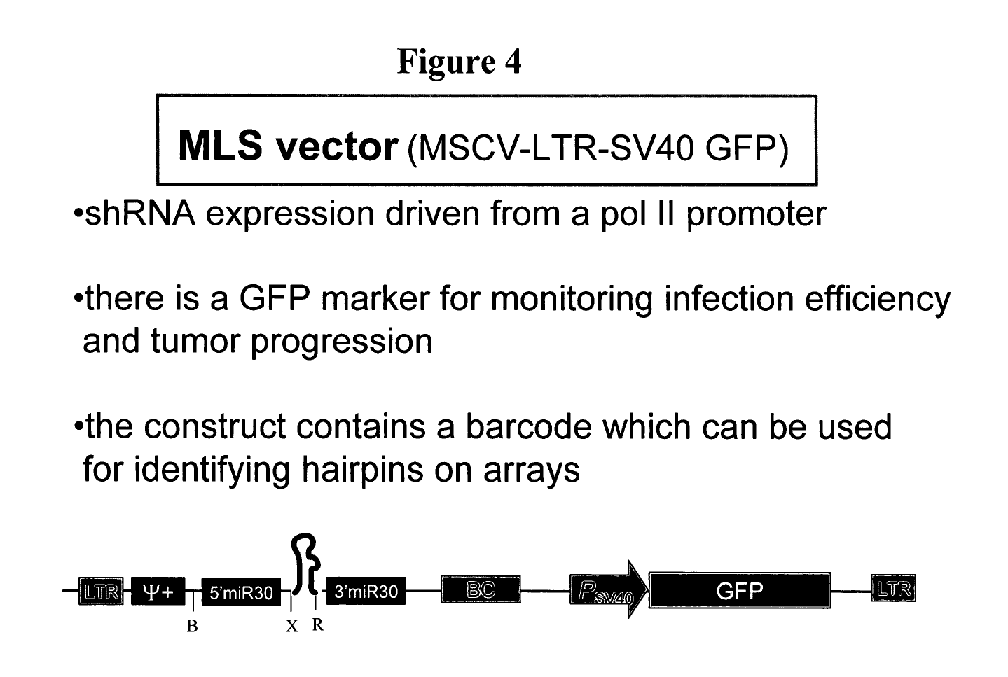

For transfection, a MLS vector was prepared as described in FIG. 4. Briefly, the vector enables shRNA expression driven from a RNA polymerase II promoter. A green fluorescent protein (GFP) is included in the construct for monitoring infection efficiency and tumor progression. For the ease of identification of specific shRNAs within the pooled analysis, the vector also comprises a bar code which allows for the measurement of the relative abundance of each individual shRNA in the population of cells infected with an entire library of shRNAs.

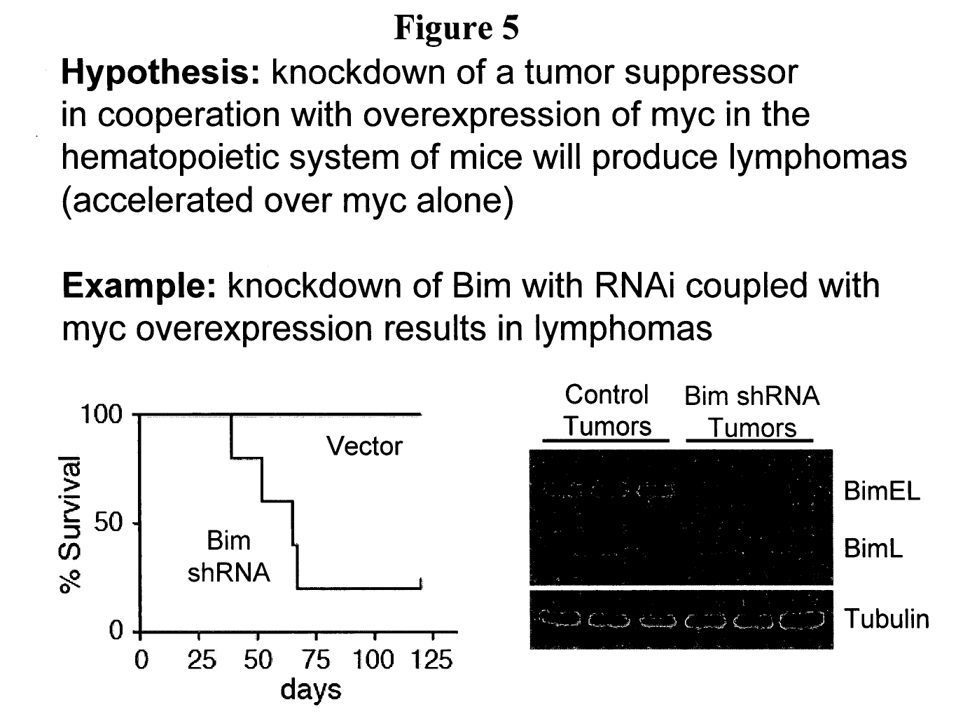

The initial infection rate was between 30 an 40%. The survival rate of infected cells clearly dropped when a tumor suppressor gene was silenced in the presence of Myc expression. FIG. 5 shows the survival rate of cells with knockdown of a tumor suppressor, Bim, with RNAi coupled with Myc over-expression. The left panel shows the survival rate of the hematopoietic cells as compared with an empty vector, used as a negative control. The right panel shows the lack of Bim in cells transfected with Bim shRNA. (Dinkins, Nature Genetics 2005). As a positive control, shRNA for p53 was introduced into the test cells at various dilutions, down to 1/100. The survival rate dropped significantly compared to vehicle control. (FIG. 6).

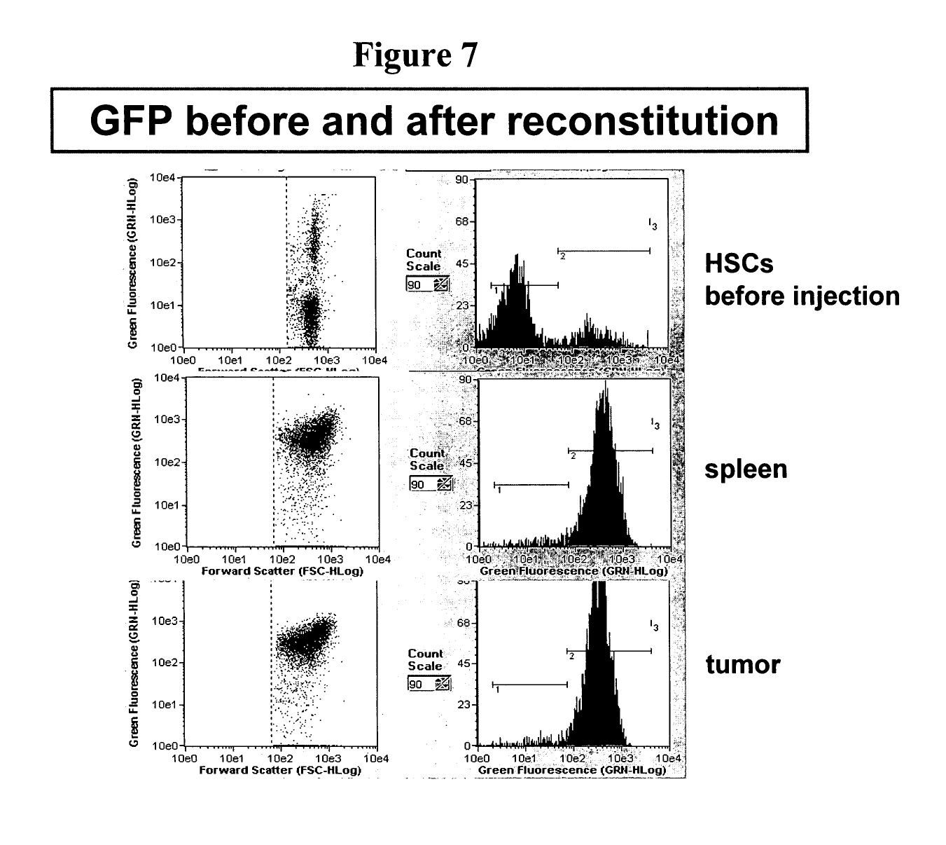

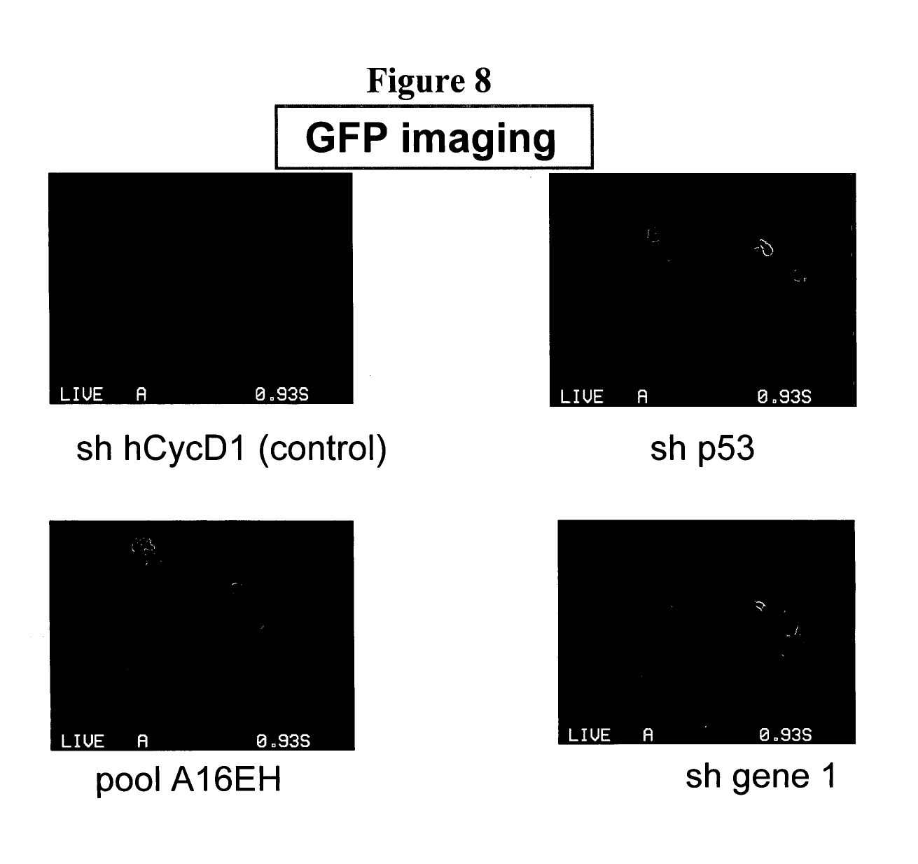

In the disclosed inventive animal model, if the presence of a shRNA in the pool confers an advantage for tumorigenesis, the mouse will develop a lymphoma. In such a case, the amount of GFP is nearly 100% in harvested spleen and tumor. See FIG. 7. In fact, a mouse spleen analyzed three weeks after injection would be green if the hairpin is advantageous. In the control vector and hairpin, the amount of GFP was about the same as it was in the stem cells. See FIG. 8.

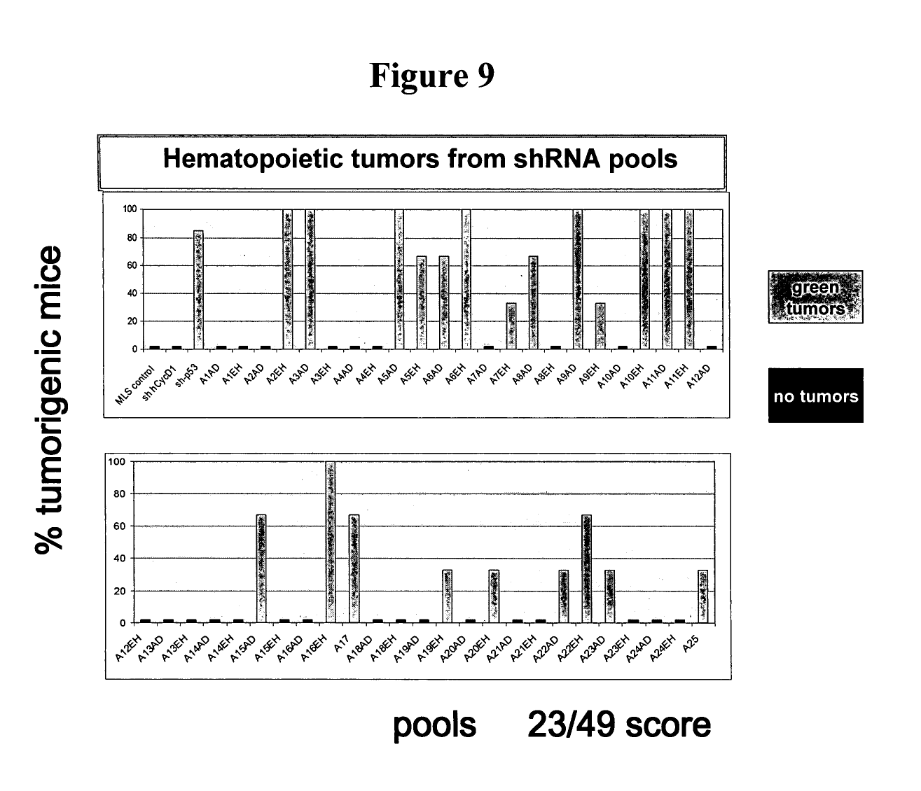

All the pools in the “Cancer 1000” library were used to reconstitute mice, and 23 of 49 pools have scored. After 16 weeks, the observation was terminated. As shown in the graph of FIG. 9, all the tumors that come up in that time were green from the presence of GFP, while no stumor resulted in the controls in that time. A total of 2352 different shRNA constructs were used. These constructs were grouped into separate “pools” of 48 shRNAs/per pool, for a total of 49 pools. One pool (containing 48 shRNAs) was introduced into 3 mice. 23 out of the 49 pools illustrated green tumors. Light bars show the % tumorigenic mice showing green tumors, and dark bars indicate no tumors. Occasional tumors were seen at a much later time, for example at about 6 months. These were not green and were therefore designated as standard Eμ-myc lymphomas unrelated to the experiment.

FIG. 10 shows the schematic for validation procedure. The shRNAs are re-introduced into HSCs and assessed for knockdown, which is done by immunoblotting or QPCR. The individual shRNAs are also reassessed in mice. To confirm involvement of the target tumor suppressor gene, new shRNAs specific to that gene are created and put back into the same mice. The new shRNAs are infected into HSCs and assessed for knockdown.

Some of the pools appear to have scored better than sh p53. However, this is a statistical artifact. There are much greater numbers for p53 because it is done as a control with every experiment. When a single pool is repeated in a larger number of mice, it does not score in every mouse, although it initially scored in 3 of 3 mice. The greatest source of variability appears to come from the stem cells, although there are always other experimental factors as well, including age, irradiation, and injection.

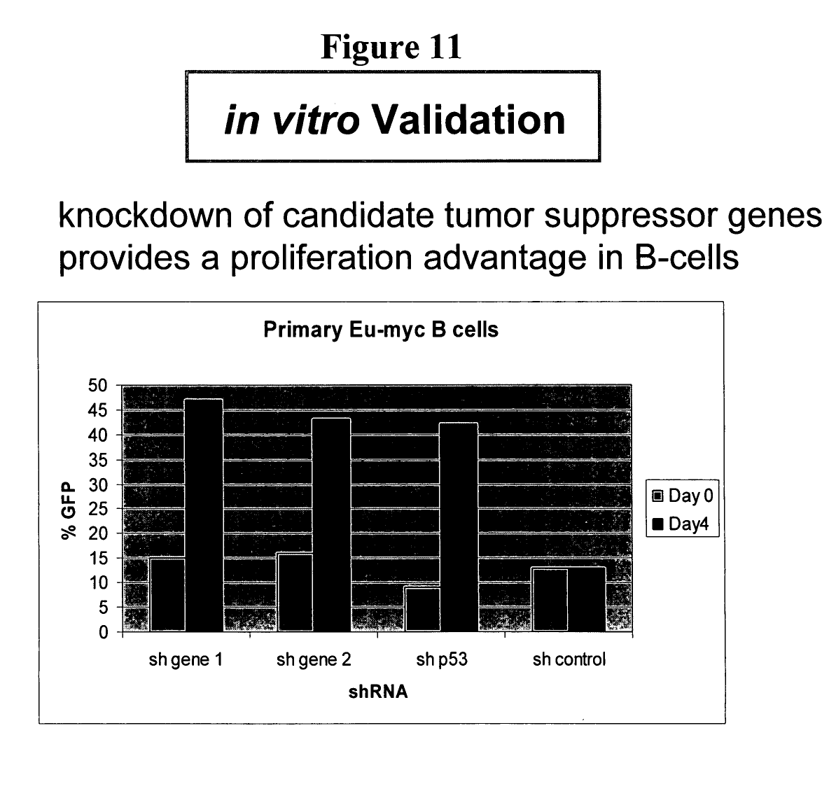

Two candidate genes were tested in the in vitro validation experiment. FIG. 11 shows that two suppressor candidates had very positive results. Knocking down these two candidates using the shRNA provided proliferation advantage to the Eμ-myc B-cells, indicating these may be tumor suppressor genes.

### Example 3

### Identified Genes Associated with Tumor Formation and/or Growth

Table I shows tumor suppressor genes identified using the method described herein. The GenBank Accession Number shows a human (except where noted) reference sequence of a cDNA for each of the identified gene. Some of the reference sequences are for the minus strand and are noted so in GenBank database. Where multiple variants are recorded, the Accession Number of the longest sequence is noted for the convenience. The invention comprises any allelic or splice variants and paralogs and xenogeneic sequences that have substantially the same biological activities as a normally functioning gene listed in Table I.

TABLE I
identified tumor suppressor genes with cDNA GenBank RefSeq Acc. No.

| | | | | |
|---|---|---|---|---|
| Mek1 | Rad17 | Angpt2 | Numb | Sfrp1 |
| NM_002755 | NM_133338 | NM_001147.2 | NM_001005743 | NM_003012 |
| SEQ ID NO: 1 | SEQ ID NO: 2 | SEQ ID NO: 3 | SEQ ID NO: 4 | SEQ ID NO: 5 |
| Fgf15 | Ppid | Shbg | Cyp1b1 | Bmp3 |
| NM_008003 | NM_005038 | NM_001040 | NM_000104 | NM_001201 |
| (mouse) | SEQ ID NO: 7 | SEQ ID NO: 8 | SEQ ID NO: 9 | SEQ ID NO: 10 |
| SEQ ID NO: 6 | | | | |
| Bag1 | Gja5 | Ngb | Arg2 | Ptpn1 |
| NM_004323 | NM_005266 | NM_021257 | NM_001172 | NM_002827 |
| SEQ ID NO: 11 | SEQ ID NO: 12 | SEQ ID NO: 13 | SEQ ID NO: 14 | SEQ ID NO: 15 |
| Edg2 | Nr2f1 | Fxyd2 | Tyms | |
| NM_001401 | NM_005654 | NM_001680 | NM_001071 | |
| SEQ ID NO: 16 | SEQ ID NO: 17 | SEQ ID NO: 18 | SEQ ID NO: 19 | |
| Nudt4 | Appbp2 | Max | Rad51c | Olfr297 |
| NM_019094.4 | NM_006380 | NM_002382 | NM_058216 | NM_146618 |
| SEQ ID NO: 20 | SEQ ID NO: 21 | SEQ ID NO: 22 | SEQ ID NO: 23 | (mouse) |
| | | | | SEQ ID NO: 24 |
| Pglyrp4 | Rarb | Fzd7 | Ctso | Tnfrs10b |
| NM_020393 | NM_000965 | NM_003507 | NM_001334 | NM_003842 |
| SEQ ID NO: 25 | SEQ ID NO: 26 | SEQ ID NO: 27 | SEQ ID NO: 28 | SEQ ID NO: 29 |
| Rhob | Orc11 | Cdk5 | Nrob1 | Ccnf |
| NM_004040 | NM_004153 | NM_004935 | NM_000475 | NM_001761 |
| SEQ ID NO: 30 | SEQ ID NO: 31 | SEQ ID NO: 32 | SEQ ID NO: 33 | SEQ ID NO: 34 |
| Spp1 | Hdac2 | | | |
| NM_001040058 | NM_001527 | | | |
| SEQ ID NO: 35 | SEQ ID NO: 36 | | | |
| Mmp7 | Prx2 | ATM | SerpinE1 | Plaur |
| NM_002423 | NM_016307 | NM_000051 | NM_000602 | NM_002659 |
| SEQ ID NO: 37 | SEQ ID NO: 38 | SEQ ID NO: 39 | SEQ ID NO: 40 | SEQ ID NO: 41 |
| Prkcb1 | Gtf2e2 | Bak1 | SerpinE2 | ATR |
| NM_002738 | NM_002095 | NM_001188 | NM_006216 | NM_001184 |
| SEQ ID NO: 42 | SEQ ID NO: 43 | SEQ ID NO: 44 | SEQ ID NO: 45 | SEQ ID NO: 46 |

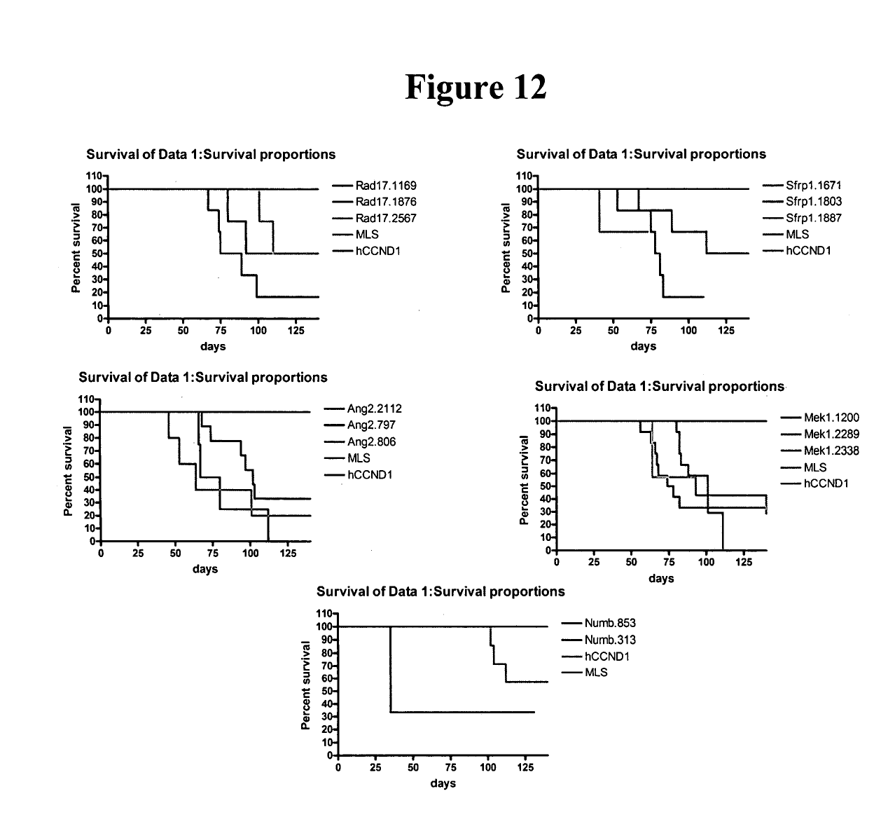

When these genes were silenced using shRNA as disclosed herein, tumors developed, indicating their roles in suppressing tumor formation and growth in a normal cell. In particular, the following five genes were of significance: Mek1; Angiopoietin2 (Ang2); Rad17; Sfrp1; Numb. The survival curves using the mouse lymphoma model, as described herein, are shown for the five genes (FIG. 12). The genes were knocked down using several different shRNA, and thus have been validated as physiologically relevant genes.

The practice of the various aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Current Protocols in Molecular Biology, by Ausubel et al., Greene Publishing Associates (1992, and Supplements to 2003); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W.H. Freeman & Co., New York (2000); Griffiths et al., Introduction to Genetic Analysis, 7th ed., W.H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All patents, patent applications and references cited throughout this disclosure are incorporated in their entirety by reference.

Those skilled in the art will recognize, or be able to ascertain using no more than routine experimentation, many equivalents to the specific embodiments of the invention described herein. Such equivalents are intended to be encompassed by the following embodiments.

1. A method of identifying a novel tumor suppressor gene comprising the steps of:
(a) obtaining a mouse comprising murine hematopoietic stem cells that overexpress Myc and have been transfected with a pool of small interfering RNA (siRNA) molecules targeting members of a library of candidate tumor suppressor genes,
(b) isolating the genomic DNA from any tumor that develops in the mouse, and
(c) identifying a nucleic acid construct that has been integrated into the genomic DNA in the tumor, wherein the identified gene of the integrated construct is a tumor suppressor gene,
thereby identifying a tumor suppressor gene that is the target of the siRNA.

2. The method according to claim 1, wherein the siRNA is short hairpin RNA (shRNA), mir-30 short hairpin RNA (shRNAmir), or microRNA (miRNA).

3. The method according to claim 1, wherein the pool of siRNA comprises nucleic acid having human target sequences.

4. A method of treating cancer in a subject comprising the steps of:
(a) determining the status in cancerous tissue in the subject of one or more tumor suppressor genes,
(b) identifying one or more target tumor suppressor genes with decreased activity in such cancerous tissue by comparing the status of such tumor suppressor gene or genes to its status in normal tissue, and
(c) increasing the activity of the target tumor suppressor(s) to restore cancerous tissue to normal tissue and
thereby treating cancer in the subject.

5. The method according to claim 4, wherein the activity of the target suppressor gene is increased by introducing into cells of the cancerous tissue an expression vector containing nucleic acid encoding the tumor suppressor gene in its full length, or a fragment, analog, or mutant thereof that encodes a physiologically active polypeptide when expressed.

6. The method according to claim 4, wherein the activity of the target suppressor gene is increased by introducing into cells of the cancerous tissue the target suppressor polypeptide in its full length, or a physiologically active fragment, analog or mutant thereof.

7. The method according to claim 4, wherein the activity of the target tumor suppressor gene is increased by modulating known upstream factors of the target tumor suppressor to increase the expression of the target tumor suppressor.

8. The method according to claim 4, wherein the activity of the target tumor suppressor gene is increased by modulating known immediate downstream factors of the target tumor suppressor to augment the activity of the tumor suppressor.

9. A method of treating cancer in a subject comprising the steps of:
(a) determining the status in cancerous tissue of one or more tumor suppressor genes,
(b) identifying one or more target tumor suppressor genes with increased or decreased activity in such cancerous tissue by comparing the status of such tumor suppressor gene or genes to its status in normal tissue, and
(c) administering to the subject a therapeutic agent known to be effective in treating such cancers that are associated with the increased or decreased activities of such gene or genes,
thereby treating such cancer in the subject.

10. A method of treating cancer in a subject comprising the steps of:
(a) determining the status in cancerous tissue of one or more tumor suppressor genes,
(b) identifying one or more target tumor suppressor genes with decreased activity in such cancerous tissue by comparing the status of such tumor suppressor gene or genes to its status in normal tissue, and
(c) administering to the subject a therapeutic agent known not to interfere with or antagonize decreased activities of such gene or genes.

11. The method according to claim 4, wherein the tumor suppressor genes are identified by:
(a) obtaining a mouse comprising murine hematopoietic stem cells that overexpress Myc and have been transfected with a pool of small interfering RNA (siRNA) molecules targeting members of a library of candidate tumor suppressor genes,
(b) isolating the genomic DNA from any tumor that develops in the mouse, and
(c) identifying a nucleic acid construct that has been integrated into the genomic DNA in the tumor, wherein the identified gene of the integrated construct is a tumor suppressor gene.

12. The method according to claim 11, wherein the tumor suppressor gene is selected from the group consisting of genes shown in Table I.

13. The method according to claim 12, wherein the tumor suppressor gene is selected from the group consisting of: MEK1; Angiopoietin 2 (Ang2); Rad17; Sfrp1; and Numb.

14. A pharmaceutical composition for the treatment of cancer in which the activity of a tumor suppressor is decreased in cancerous tissue compared to such activity in normal tissue, comprising an expression vector containing the tumor suppressor gene in its full length or a fragment, analog, or mutant thereof that encodes a physiologically active polypeptide.

15. A pharmaceutical composition for the treatment of cancer in which the activity of a tumor suppressor is decreased in cancerous tissue compared to such activity in normal tissue, comprising the tumor suppressor protein or a physiologically active fragment, analog, or mutant thereof.

16. A pharmaceutical composition for the treatment of cancer in which the activity of a tumor suppressor is decreased or increased in cancerous tissue compared to such activity in normal tissue, comprising one or more therapeutic agents that modulate known upstream factors of the tumor suppressor to increase or decrease toward normal the tumor suppressor expression the activity of the tumor suppressor.

17. A pharmaceutical composition for the treatment of cancer in which the activity of a tumor suppressor is decreased or increased in cancerous tissue compared to such activity in normal tissue, comprising one or more therapeutic agents that modulate known immediate downstream factors of the tumor suppressor to increase or decrease toward normal the tumor suppressor expression.

18. The pharmaceutical composition of claim 14, wherein the tumor suppressor gene is selected from genes shown in Table I.

19. The pharmaceutical composition of claim 17, wherein the tumor suppressor gene is selected from the group consisting of MEK1; Angiopoietin 2 (Ang2); Rad17; Sfrp1; and Numb.

20. A method for identifying a therapeutic agent effective to treat cancer, comprising the steps of:
(a) contacting a candidate therapeutic agent with a mouse lymphoma having a genome comprising a myc gene operably linked to an Eμ-IgH enhancer and further comprising shRNA of a tumor suppressor gene; and
(b) monitoring the mouse for remission of the lymphoma, wherein remission of the lymphoma indicates the effectiveness of the candidate therapeutic agent,
thereby identifying a therapeutic agent.

21. A method for identifying a therapeutic agent effective to treat cancer, comprising the steps of:
(a) contacting a candidate therapeutic agent in vitro with cells derived from mouse lymphoma having a genome comprising a myc gene operably linked to an Eμ-IgH enhancer and further comprising shRNA of a tumor suppressor gene; and
(b) monitoring the cells for growth, wherein slowing or arresting of growth indicates the effectiveness of the candidate therapeutic agent,
thereby identifying a therapeutic agent.

22. The method of claim 20, wherein the tumor suppressor gene is selected from genes shown in Table I.

23. The method of claim 22, wherein the tumor suppressor gene is selected from the group consisting of MEK1; Angiopoietin 2 (Ang2); Rad17; Sfrp1; and Numb.

24. A method of diagnosing a cancer in a subject, comprising obtaining a tissue sample from the subject, determining the biological activity of one or more tumor suppressor selected from those shown in Table I in the tissue sample and comparing said activity to that in normal tissue, wherein the subject is diagnosed with cancer if the activity of any one of tumor suppressor is substantially decreased or is not detectable in the tissue sample.

25. A method of diagnosing a cancer in a subject, comprising obtaining a tissue sample from the subject, determining the expression of one or more tumor suppressor gene selected from genes shown in Table I in the tissue sample and comparing said expression to that in normal tissue, wherein the subject is diagnosed with cancer if said expression is substantially decreased or no expression is detected in the tissue sample.
